(19) United States
(12) Patent Application Publication
(10) Pub. No.: US 2007/0156089 A1
(43) Pub. Date: Jul. 5, 2007

(54) Multi-channel rotary peristaltic infusion pump

(76) Inventor: George Yu, Flushing, NY (US)

Correspondence Address: GEORGE YU, 153-28 SANFORD AVENUE, FLUSHING, NY, 11355, US

(21) Appl. No.: 11/320,933

(22) Filed: Dec. 29, 2005

(51) Int. Cl. A61M 37/00

(52) U.S. Cl. 604/131

(57) ABSTRACT

Multi-channel IV therapies are achieved through the use of a single source infusion pump, which incorporates three rotary chambers. Each rotary chamber is composed of a base panel with the appropriate cavity for housing a rotor with three sets of rollers, plus tubing defined pathway going into and out of the rotor cavity. Components such as a knob and a sliding latch are incorporated into the inflow and outflow pathways on the base panel respectively to accommodate the installation and removal of anti-free flow disposable IV pump sets. These features also ensures that a properly installed anti-free flow IV pump set will remain secured in the base panel, even when mechanical impulses are experienced by the pump. The chamber tubing of the anti-free flow disposable IV pump set is composed of thin-walled rubber tubing that connects to an inflow stopcock and terminates through outflow clear connector. Fluid within the installed chamber tubing is pushed forward as the rotor rotates and its rollers roll and compress onto the rubber tubing against the wall of the cavity. A force sensor is incorporated in the inflow pathway on the base panel to detect upstream occlusion in the IV pump set, and an air detection system is incorporated in the outflow pathway on the base panel to detect the presence of air within clear connector.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING

Not Applicable

### BACKGROUND OF THE INVENTION

1) Field of Invention

This invention relates to the rotary peristaltic infusion pumps used in the health care industries, and more specifically to the use of single source infusion pump capable of administrating up to three IV fluids to the patient. The use of this versatile infusion device is often referred to as the “Three-Channel Infusion Pump” or “Multi-Channel Infusion Pump”. Alaris Medical System, Inc. is a well-known manufacturer that has developed the “Med System III Infusion System”. The system incorporates three piston drives that uses up to three independent disposable cassette-type IV pump sets for its fluid administration applications.

Another manufacturer that has developed the multi-channel infusion pump is Baxter Healthcare Corporation. Their latest infusion device is the “Colleague CX Volumetric Infusion Pump”. The pump utilizes three complex linear drives for its fluid delivery system. Each drive provides the fingers-like action by pressing onto the chamber portion of the independent disposable IV pump set that has been installed onto the pump's fluid chamber. The linear action generated by the drive pushes the fluids forward through the disposable IV pump set, within controlled flow rate.

The introduction of the multi-channel infusion pumps has further facilitated the IV therapy in many ways. Foremost, due to the pump's multi IV fluid delivery system, the hospital staff only needs to interface with one infusion pump instead of many. The second advantage is space consideration. Stacking multiple infusion pumps on the IV pole or bedside clamp takes the space away from attaching other medical devices. In addition, as the number of medical equipment usage increases, the chance of patient's over exposure to wires also increases.

Even with the modern advances of the current multi-channel infusion pumps, certain drawbacks have been reported with the use of these devices. A simple bump or fall of the equipment onto the ground, often engaged accidentally by the person handling these devices may cause damage to the mechanical drive of the infusion pump. Equipments often handled by the paramedics during an emergency patient transport take a lot of punishment, mostly due to the harsh road conditions. This factor reduces the effectiveness of the current multi-channel infusion pumps and increases risks for the patients.

Another drawback found on some of the current multi-channel pumps occurs when the infusion pump is accidentally turned upside down while it is administrating fluid medication. The pump using the cassette-type IV pump set will accumulate large quantities of air bubbles in its cassette chamber, thus posing risks to the patients.

On the other hand, the application of rotary peristaltic infusion pumps has a history of accurate performance and reliability. The pump chamber simply utilizes a rotor, which is composed of a circular shaped body with a pair of rollers mounted opposed to each other on the edge of rotor's body. An independent IV pump set is installed onto the fluid tubing path within the pump chamber. As the rotor rotates by means of a motor driven source, the rollers roll and exert sufficient compressive force onto the chamber fluid tubing, thus pushing the fluid forward through the IV pump set. The chamber fluid tubing is usually made of silicone rubber. The rotary drive system provides a cost effective solution by keeping the system components simple. Also, the incorporation of modern day plastic into the rotary system enhances the pump's abilities to handle the extreme conditions. Even when the pump is accidentally turned upside down, it will still maintain proper functioning.

Currently the rotary peristaltic infusion pumps used for IV therapy use only one rotary chamber per pump. Certain safety issues have arisen with the use of these pumps that limited their use in various hospital organizations. One safety issue, which involves the pump's inability to detect upstream occlusion, was addressed in the report of Hazard [Health Devices June 1986; 15(6); 182-4]. The article discusses that certain infusion pumps continue to operate and falsely alert the hospital staff of the completion of drug therapy, even when no fluid was administrated from the pump. The problem was due to a fluid restriction in the upstream line of the IV pump set. This posed a dangerous threat for the patient, and thus the upstream occlusion detection system became a mandatory safety measure for infusion pumps.

Another safety issue addressed to certain infusion pumps was the lack of anti-free flow device incorporation. An article dating back to Apr. 22, 1998 from the ISMP publication of Medication Safety Alert discusses that when IV pump set is temporary removed from the infusion pump without clamping it first, and while it remain attached to the patient IV line, gravity forces the remaining fluid from the IV bag to free-flow through the unclamped IV pump set into the patient's vein, thus causing drug overdose. This type of accident is often fatal to the patient, and therefore, it is mandatory for infusion pumps to incorporate the use of independent anti-free flow IV pump set.

By incorporating all the safety requirements, along with the advantages of the rotary peristaltic infusion device into the multi-channel infusion pump, the multi-channel infusion pump disclosed hereon within these claims will provide more benefits to the medical society.

2) Prior Knowledge

The following table is a list of the patent documents, which contain information on the rotary peristaltic infusion pumps.

| Patent Reference #: | Date: | Name: |
|---|---|---|
| 4,184,815 | January 1980 | Casson et al. |
| 4,798,590 | January 1989 | O'Leary et al. |
| 6,102,678 | August 2000 | Peclat |

Reference documents containing information regarding the safety measure standards, such as anti-free flow IV pump sets, upstream occlusion detection systems, or air bubbles detection in tubing are listed in the following table.

| Patent Reference #: | Date: | Name: |
|---|---|---|
| 5,616,124 | April 1997 | Hague et al. |
| 5,788,674 | August 1998 | McWilliams |
| 6,358,225 B1 | March 2002 | Butterfield |

### SUMMARY OF THE INVENTION

The primary objective of this invention is to provide an infusion pump with multi-channel infusion capabilities, thereby incorporating three rotary chambers, which exhibits the advantages of the rotary peristaltic infusion pumps.

A further objective of this invention is to provide such a pump that each of its rotary chamber consists of a base panel with appropriate cavity for housing the rotor assembly, plus tubing defined pathway going into and out of the cavity, which accommodates the installation and removal of the anti-free flow disposable IV pump set.

A further objective of this invention is to provide an anti-free flow disposable IV pump set with its chamber tubing composed of thin-walled rubber tubing that connects to an inflow stopcock, and terminates through an outflow clear connector. The length of the thin-walled rubber tubing is sized for optimum fluid transport efficiency.

A still further objective of this invention is to provide a rotor assembly that consists of a circular shaped body with three flexible arms spaced 120 degrees apart, and each flexible arm supports a roller, thereby as the rotor rotates, the rollers exert sufficient compressive force onto the chamber rubber tubing, resulting in fluid being pushed through the IV pump set and delivered to the patient at precise flow rates.

A yet further objective of this invention is to provide means of detecting the upstream occlusion by installing a force sensor at the inflow tubing pathway on the base panel, and to provide means of detecting the air bubbles through the clear connector of the IV pump set by installing an infrared air detection system in the outflow tubing pathway on the base panel.

The preferred form of this invention includes the incorporation of three rotary chambers onto a single unit infusion pump, which uses independent anti-free flow disposable IV pump sets to provide multiple intravenous therapies to the patient. The chamber tubing of the IV pump set is made of rubber tubing that connects from inflow stopcock through an outflow clear connector, which is looped through the tubing defined pathway going into and out of the rotor cavity on the base of the rotary chamber. As the rotor rotates by means of a motor driven source, the rollers on the rotor exert sufficient compressive force onto the chamber rubber tubing against the wall of the rotor cavity, resulting in fluid being pushed forward through the IV pump set and delivered to the patient. A force sensor mounted in the inflow-tubing pathway of the base provides upstream occlusion detection, and an air sensing system mounted on the outflow-tubing pathway of the base provides air bubble detection in the outflow clear connector of the anti-free flow disposable IV pump set.

### DESCRIPTION OF THE DRAWINGS

The features and functionalities of this invention could best be illustrated by reference drawings and they are:

FIG. 1 is a schematic view of the multi-channel rotary peristaltic infusion pump system, which also illustrates the properly installed anti-free flow disposable IV pump sets;

FIG. 2 is a partial view of the infusion pump in FIG. 1, showing its LCD screen with individual setting for channel-3 of the rotary system;

FIG. 3 is a schematic view of the anti-free flow disposable IV pump set system;

FIG. 4 is a exploded sectional view taking in perspective of FIG. 3 to show the chamber tubing of the anti-free flow disposable IV pump set;

FIG. 5 is an exploded view of the pump from FIG. 1, showing the rotary system and the seating of chamber portion of the anti-free flow disposable IV pump set;

FIG. 6 is the right side view of the rotary system from FIG. 5, showing the unlocked components on the base panel;

FIG. 7 is a cross sectional side view taking form FIG. 5 that shows the rotor assembly with rollers and the compressed chamber rubber tubing within the wall of the rotor cavity on the base panel.

FIG. 8 is an exploded partial view from FIG. 5, showing the upstream occlusion effect on the chamber rubber tubing of the anti-free flow disposable IV pump set and the occlusion detector system on the inflow pathway of the base panel.

### DETAILED DESCRIPTION OF THE INVENTION

Basic Pump Operation

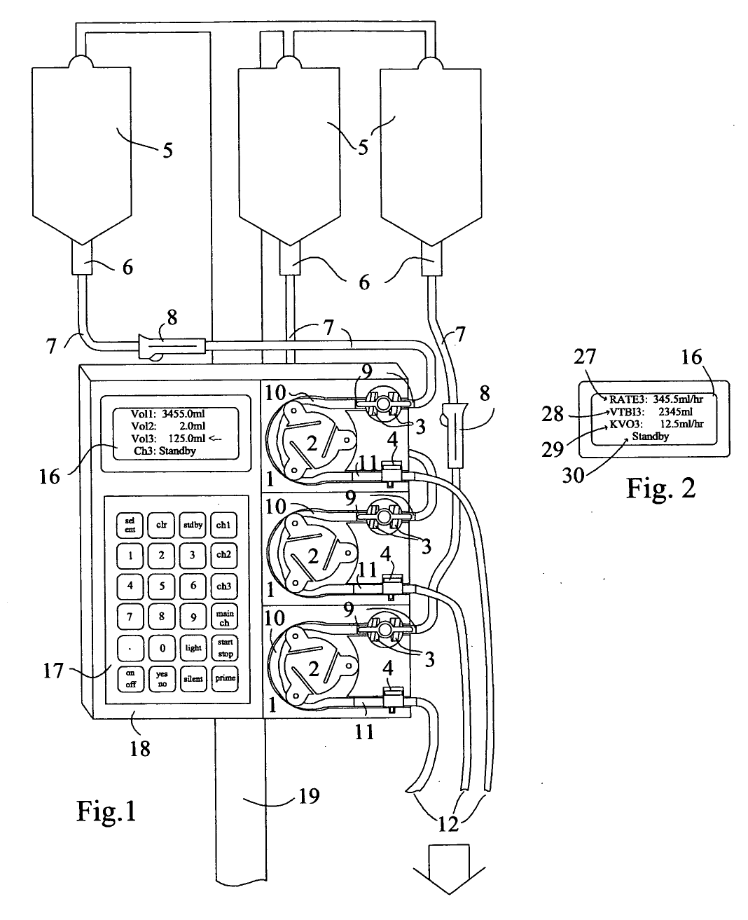

FIG. 1 illustrates the multi-channel peristaltic infusion pump 18 in accordance with this invention. Fluids from the IV bags 5 are delivered from the pump 18 by means of the anti-free flow disposable IV pump sets, usually referenced by numeral 7 to the patient line (not shown). The pump 18 incorporates three rotary chambers aligned vertically on the right side of the pump 18, referenced by numeral 1 and usually referred to as the base panel. The base panel 1 includes the appropriate cavity for mounting a rotor 2. Tubing defined pathways going into and out from the cavity are provided, such that the inflow-tubing pathway mounts a rotating knob 3 and the outflow-tubing pathway mounts a sliding latch 4, which provide ease of installation and removal of the anti-free flow disposable IV pump set 7. Electronic controls of the pump are achieved through navigating the display settings on the LCD screen 16 by the user interface keypad 17. In addition, the pump 18 has the capability to be mounted onto the IV pole stand 19.

FIG. 1 also illustrates the proper form of installed anti-free flow disposable IV pump set 7, such that the rotating knob 3 on each panel 1 is turned horizontally and the sliding latch 4 is closed. Pressing the “on/off” switch on the keypad 17 turns on the pump 18, which brings up the main setting on the LCD screen 16. The main setting displays the previous recording of infused volume from each channel, which served as history reference. An arrow sign designates the current interactive channel, whose status is displayed on the bottom of the LCD screen 16. The standby mode is the preset mode for all channels, which allows the priming of anti-free flow disposable IV pump set 7. The “prime” key on the keypad 17 enables the rotor 2 to rotate at a preset time frame so air bubbles from within the tubing are completely removed. Only a fully primed anti-free flow disposable IV pump set 7 can be connected to the patient line. Selecting an interactive channel can be accomplished by pressing the “sel” key on the keypad 17, as this function moves the arrow from channel 1 (Vol1) through channel 3 (Vol3). The “clr” key on the keypad 17 resets the total infused volume to 0.0 ml for the current interactive channel. This feature allows new fluid therapy to take place.

Pressing “ch1”, “ch2”, or “ch3” keys on the keypad 17 brings up individual settings for each channel. Referring to FIG. 2, the LCD screen 16 shows the channel 3 settings for adjusting the rate of infusion “RATE3” 27, volume to be infused “VTBI3” 28, and keep vein open “KVO3” 29. The status of channel 3 is shown on the bottom of the LCD screen 16, which currently is on “Standby” 30. The accepted ranges for the rate of infusion are from 0.1 ml/hr to 999.9 ml/hr. The accepted ranges for the volume to be infused are from 1 ml to 9999 ml, and the ranges for keep vein open are from 0.1 ml/hr to 19.9 ml/hr.

After the prescribed variables have been entered for the interactive channel, pressing the “start/stop” key on the keypad 17 enables the current interactive channel to start infusing fluid medication. In addition, the word “Infusing” is displayed on the bottom of the LCD 16 to indicate the status of the current channel. Pressing the “start/stop” key again puts the current interactive channel of the pump 18 into temporary stop mode, and the word “Stopped” is displayed on the bottom of the LCD screen 16 of FIG. 2. This function allows the user to make adjustments on the pump 18. For safety purpose, the stop mode is a timer-based mode that alerts users with a tone, to resume pump 18 operation. The “silent” option on the keypad 17 provides the user more time by silencing the tone and holding off the stop mode for a period of time.

In order to view the infused volume on each channel, the “main ch” key on the keypad 17 brings back the main setting on the LCD screen 16 of FIG. 1. If any of the channels engages a problem during operation, the affected channel automatically stops. The arrow sign will automatically points to the affected channel, followed by the display of its alarm condition on the bottom of the LCD screen 16 with pulsed tone. The user is prompted to take the appropriate action, and the pump 18 will resume its normal operation by the “start/stop” key on the keypad 17.

Rotary System

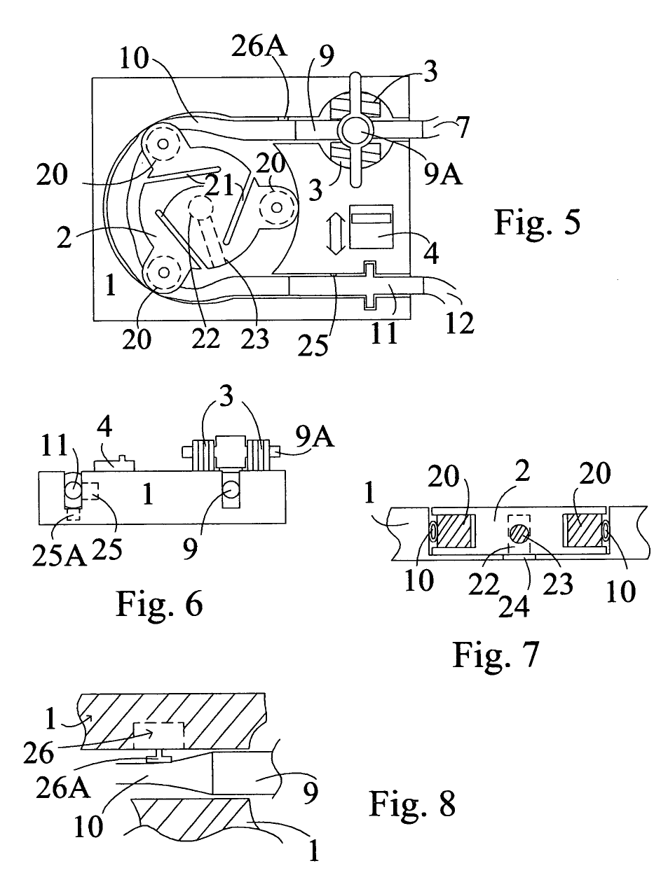

Referring to FIG. 5 and FIG. 7, the rotor 2 is composed of a circular shaped body with three protruding arms that are spaced 120 degrees apart, and each arm of the rotor 2 holds a roller 20. Three cuts 21 are made equidistance into the body of the rotor 2 to make the arms of the rotor 2 flexible. This allows the accommodation of various tolerances experienced on the wall thickness of the chamber rubber tubing 10 during manufacturing process, temperature effect, fluid viscosity, and hours of usage. Modern day plastic also allows the rotor 2 to overcome the backpressure of IV pump set 7 during the infusion process.

The drive of the rotor 2, preferable a motor source (not shown) is mounted on back of the base panel 1. The base panel 1 provides a shafting hole 24, which aligns with the center of the rotor 2 that allows the motor's cylindrical shaft (not shown) to go through. The rotor 2 mounts onto the motor's cylindrical shaft from the drilled hole 22 on its backside, and is secured by the screw through its side-tapped hole 23.

When the pump 18 is set to infuse, the rotor 2 starts to rotate counter-clockwise, which the rollers 20 of the rotor 2 roll and exert sufficient compressive force onto the chamber rubber tubing 10 against the wall of the cavity on the base panel 1. The action pushes the fluid forward through the IV pump set 7 towards the patient line. In addition, three rollers 20 on the rotor provide smoother fluid delivery to the patient.

Independent Anti-Free Flow Disposable IV Pump Set

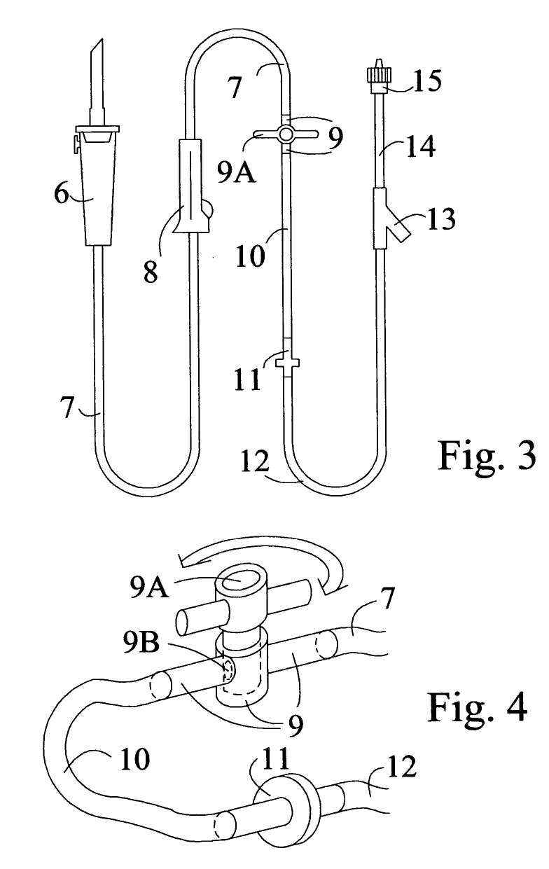

Referring to FIG. 3, the anti-free flow disposable IV pump set 7 illustrated is in accordance with the disclosed pump system. The IV pump set 7 is made of the vented spike drip chamber 6 that inserts into the IV source such as the IV bags 5 of FIG. 1. Fluid flows form the vented spike drip chamber 6 into the upstream line tubing 7, which is usually referred as the “pump set”. The upstream line tubing 7 includes a roller clamp 8 for restricting fluid flow during pump set 7 removal from the pump 18, and is connected to an inflow stopcock 9, which provides anti-free flow system for the pump set 7.

Referring to FIG. 4, the stopcock 9 includes a handle 9A with an opening valve 9B. When the handle 9A is aligned with the stopcock's body 9, the valve 9B is facing the opening pathway of the stopcock 9. This position allows fluid to flow through. If the handle 9A is turned 90 degrees in any direction, away from the opening pathway of the stopcock's body 9, the valve 9B faces the ridged body of the stopcock 9, which restricts fluids from flowing through. The rotation on the handle 9A served as the anti-free flow system for the pump set 7, and the pump 18 of FIG. 1 incorporates a knob 3 on its panel 1 that accommodates this system.

The chamber tubing is composed of thin-walled translucent rubber tubing 10 that connects form the inflow stopcock 9 to the outflow clear connector 11. These components are installed onto the pump 18 of FIG. 1, and they are illustrated in detail in FIG. 4. The rubber tubing 10 provides the flexibility and durability to be compressed by the rollers 20 of FIG. 5. The outflow clear connector 11 connects to the downstream line tubing 12, which connects to a Y-site 13 and terminates to the end line tubing 14 that is coupled to the spin lock male connector 15.

Installation and Removal of the Anti-Free Flow Disposable IV Pump Set

Referring to FIG. 5 and FIG. 6, the installation of the IV pump set 7 involves inserting the stopcock 9, whose handle 9A is originally turned 90 degrees away from its body, into the slot of the vertically positioned knob 3 on the base panel 1. The chamber rubber tubing 10 is looped through the rotor 2 by rotating the rotor 2 counter-clockwise, and the outflow clear connector 11 is placed into the outflow port of the base panel 1. With the proper seating of the chamber tubing, the sliding latch 4 is moved downward to close the outflow tubing port, while the knob 3 is rotated 90 degrees counter-clockwise, which locks the stopcock 9 onto the base panel 1 and aligns the stopcock's valve 9B of FIG. 4 to the opening connection of the stopcock's body 9. The compressive rollers 20 prevent fluids to free-flow through the IV pump set 7. Any infusion engaged by the pump 18 of FIG. 1 at this time enables fluid to be delivered through the IV pump set 7 to the patient. The removal of the IV pump set 7 involves stopping the current interactive channel and reversing the procedures for installation of IV pump set 7.

Upstream Occlusion Detection System

Referring to FIG. 8, when the pump experienced a fluid restriction in the upstream line of its pump set 7 while the rotor 2 of FIG. 1 continues to rotate, the chamber rubber tubing 10 will immediately collapse from the resulting action. A force sensor 26 with spring like plate 26A is installed in the inflow tubing pathway of the base panel 1 to detect the upstream occlusion phenomenon. An ideal location of the spring plate 26A is shown in FIG. 5. As the chamber rubber tubing 10 collapses, the spring plate 26A pushes outward away from its force sensor 26, which results in a signal being sent to the CPU controller of the pump 18 in FIG. 1 as upstream occlusion alarm. The affected channel immediately stops, and the appropriate alarm will alert the user of its condition and proper action to be taken. Under normal operating condition, the chamber rubber tubing 10 stays firm and pushes the spring plate 26A inward toward the force sensor 26, which the resulting signal is interpreted as satisfactory condition.

Air Bubble Detection System

Referring to FIG. 5 and FIG. 6, the air bubble detector is composed of an infrared transmitter 25 and an infrared receiver 25A, which are installed on the outflow pathway of the base panel 1. The infrared transmitter 25 sends an infrared signal through the outflow clear connector 11. The medium within the outflow clear connector 11 affects the outcome of the signal that will be sent to the CPU controller. The presence of fluid within the outflow clear connector 11 lowers the intensity of the infrared signal, which is insufficient to trigger the infrared receiver 25A. The presence of air within the outflow clear connector 11 raises the intensity of the infrared signal and triggers the infrared receiver 25A, thus the resulting signal is sent to CPU controller and translated as air in line alarm.

By drawing the benefits of the rotary peristaltic infusion pumps and incorporating the safety measures of anti-free flow disposable IV pump sets, upstream occlusion detection system, and air bubble detection system, in addition to reduce the complex mechanical drive systems featured on the current multi-channel infusion pumps, the presented invention here will further facilitate the healthcare industry.

1. A single unit infusion pump that incorporates three rotary chambers, and each said rotary chamber is composed of a rectangular shaped panel with the appropriate cavity for housing a rotor with circular shaped body and flexible three arms that are spaced 120 degrees apart, and each said arm holds a roller, which as the said rotor rotates by means of a motor driven source, the said rollers exert sufficient compressive force onto the installed chamber tubing of the anti-free flow disposable IV pump set, resulting in fluid being delivered from the IV source to the patient.

2. The pump of claim 1, in addition that the said rectangular shaped panel includes a tubing pathway that dictates the inflow and outflow into the said rotor cavity, whereby the said inflow pathway incorporates a seating/knob and the said outflow pathway incorporates a sliding latch, which accommodates the ease of installation and removal of said anti-free flow disposable IV pump set of claim 1.

3. The anti-free flow disposable IV pump set of claim 1, which the said chamber tubing is composed of thin-walled translucent rubber sized for optimum fluid transport that connects form an inflow stopcock and terminates through an outflow clear connector.

4. The pump of claim 2, in addition that said inflow tubing pathway of the said panel incorporates a force sensor to detect the upstream occlusion effect experienced on the said anti-free flow disposable IV pump set.

5. The pump of claim 4, in addition that said outflow-tubing pathway of the said panel incorporates an air bubble detector system to detect the presence of air within the said clear connector of claim 3.
